(19) United States
(12) Patent Application Publication
(10) Pub. No.: US 2025/0354106 A1
(43) Pub. Date: Nov. 20, 2025

(54) SAMPLE VESSEL FOR CULTIVATING BIOLOGICAL SAMPLES, APPARATUS FOR OPERATION THEREOF, AND MICROSCOPE

(71) Applicant: Carl Zeiss AG, Oberkochen (DE)

(72) Inventors: Anne Wuttke, Dresden (DE); Matthias Eibl, Regensburg (DE)

(21) Appl. No.: 19/290,098

(22) Filed: Aug. 4, 2025

Related U.S. Application Data
(63) Continuation of application No. PCT/EP2024/052523, filed on Feb. 1, 2024.

(30) Foreign Application Priority Data
Feb. 2, 2023 (DE) 10 2023 200 837.8

(51) Int. Cl. C12M 1/00; C12M 1/32; C12M 1/34

(52) U.S. Cl. CPC C12M 41/06; C12M 23/12; C12M 23/22; C12M 29/04; C12M 29/14; C12M 41/40

(57) ABSTRACT

A sample vessel is for cultivating biological samples and includes a cavity for accommodating a medium; at least one access opening for delivering the medium into the cavity; and a sample space, disposed within the cavity, for accommodating a sample. The sample space being separated from a remaining space of the cavity by at least one lateral wall having apertures via which the medium in the cavity can communicate with the sample space. The at least one lateral wall stands on a bottom of the sample space; and the bottom is transmissive for wavelengths of at least one wavelength range of visible light, such that illumination of the sample space and/or detection of detection radiation coming out of the sample space through the bottom of the cavity is made possible. An apparatus is for operating the sample vessel and to a microscope.

### CROSS-REFERENCE TO RELATED APPLICATIONS

This application is a continuation application of international patent application PCT/EP2024/052523, filed Feb. 1, 2024, designating the United States and claiming priority from German application 10 2023 200 837.8, filed Feb. 2, 2023, and the entire content of both applications is incorporated herein by reference.

### TECHNICAL FIELD

The disclosure relates to a sample vessel for cultivating biological samples and to an apparatus for operating the sample vessel and to a microscope having such an apparatus.

### BACKGROUND

Besides the cultivation of substantially planar cell cultures (2D cell cultures), there is increasing importance in the cultivation of three-dimensional biological objects (3D cell cultures, 3D culture), for example organoids and spheroids. In an environment that allows 3D cultivation, the spatial extent of biological tissues can usually be taken into account. The biological objects produced via an in vitro 3D culture (also referred to hereinafter as “biological sample”, or “sample” for short) can adhere to a surface, rest on such a surface, be embedded in a gel-like matrix (such as Matrigel and related products), or float freely in a surrounding medium (“free-floating”).

The spatial extent of the biological objects and the residence thereof in the surrounding medium pose considerable technical challenges if the entire medium or a portion thereof is to be replaced. The purpose of such replacement is to deliver nutrients, basic building blocks for (protein) biosynthesis, and signaling substances, and to supply oxygen and remove metabolites and spent medium. If the medium is replaced by using, for example, pipette tips, they may inadvertently touch or damage the sample. Moreover, samples floating freely in the medium may be inadvertently swirled or even co-aspirated.

In contrast to 2D cell cultures, a medium change is therefore usually performed manually in 3D cultures, but this is quite elaborate. A sample-friendly medium change is particularly important for in vitro 3D cultures, since they have to be cultivated over a long period of time. Any interference can have adverse effects on the development and quality of the sample.

Known from the prior art for 2D cell cultures are ways of (semi)automatically changing the medium used. However, they are configured especially for adherent 2D cell cultures, which cannot be directly applied to free-floating samples of a 3D culture. If, on the other hand, the sample of a 3D culture is cultivated in a Matrigel drop, then although the sample is localized, the Matrigel drop may differ in height and occupy a large portion of a sample vessel, for example a well of a (micro)titer plate. This carries the risk of the sample being damaged by the pipette tip.

In order to reduce the abovementioned risks for the sample, a medium change can be carried out by carefully tipping the sample vessel or by pipetting with a large excess of medium. In both cases, a significant proportion of the medium remains in the sample vessel.

Also known are optimized culture plates that minimize the volume for the sample, thus facilitating medium exchange. An example thereof is a multiwell plate from Insphero AG, Schlieren, Switzerland (Akura™ 96 Spheroid Microplate). Formed in each well serving as a sample vessel is a channel that narrows toward the bottom of the sample vessel. As a result of this narrowing, a region of sample residence is defined and a pipette can be moved to a defined position in the well without touching the sample. However, this plate is of only limited suitability for cultures in Matrigel. For cultures requiring a larger surface area for growth, the plate is incompatible.

In order to perform an automatic medium change, slides, such as the Fluidic 480and Fluidic 983 chips from Chipshop, Jena, Germany, can be connected to pumps. However, such a configuration is incompatible with Matrigel and is only suitable for relatively small 3D cell cultures. Furthermore, scaling-up is difficult, since each individual slide has to be equipped with its own pump.

### SUMMARY

It is an object of the disclosure to provide a way of cultivating and optically detecting 3D cell cultures that is improved over the prior art.

The object is achieved by various embodiments according to the disclosure.

The object is achieved by a sample vessel for cultivating biological samples, including a cavity for accommodating a medium; at least one access opening for delivering the medium into the cavity; and at least one sample space, disposed within the cavity, for accommodating a sample, the sample space being separated from a remaining space of the cavity by at least one lateral wall. The at least one lateral wall has apertures via which a medium present in the cavity can communicate with the sample space. The medium can touch another medium present in the sample space or can enter and, for example, flow through the sample space.

A sample vessel according to the disclosure is characterized in that the at least one lateral wall stands on a bottom of the sample space. In addition, the bottom is transmissive (transparent) for wavelengths of at least one wavelength range of visible and/or infrared light, such that illumination of the sample space and/or detection of detection radiation coming out of the sample space through the bottom of the cavity is made possible.

In further embodiments of the sample vessel according to the disclosure, besides the at least one lateral wall, a bottom of the sample space may be provided with apertures via which a medium present in the cavity can communicate with the sample space.

A basic idea behind the disclosure is to divide the cavity physically and functionally in such a way that the sample is localized in one region, while, for example, a pipette tip can be used at another location in the cavity without endangering the sample. In contrast to the prior art, the sample is moreover protected from being inadvertently flushed away, since the sample is positioned in the sample space and high flow rates or a high transfer capacity can be advantageously avoided by suitably choosing the number, size and arrangement of the apertures. Moreover, the sample vessel according to the disclosure allows optical detection, visual monitoring and/or visual representation of the processes in the sample vessel.

In order to achieve the advantage outlined above, the apertures have a maximum internal width of at most 1000 μm, advantageously of at most 500 μm and preferably of at most 200 μm. In further embodiments of the sample vessel, the apertures may have internal widths of less than 200 μm, for example 100 μm or 50 μm. In order to hold individual cells in the sample space, the apertures may have internal widths of at most 10 μm.

A lateral wall of the sample space may have apertures of differing internal width. For instance, in an embodiment of the sample vessel according to the disclosure, the internal width of the apertures may become larger with increasing distance from the bottom of the sample space. This makes it possible to minimize the fluidic stress on a sample in the region of its residence, without compromising the supply of, for example, oxygen and/or nutrients to the sample.

The lateral wall(s) used may be planar structures provided with apertures, bars arranged close to one another and/or grids. The cross-section of the sample space, in plan view, may be circular, oval, polygonal or semicircular.

In an embodiment of the disclosure, the sample vessel is open at the top when it is oriented in the use state. Optionally, it may be fully or partly provided with a lid in order to reduce the risk of contamination and unintended evaporation of the contents of the cavity. The lid may be removable or have an opening that can be optionally reclosable. The access opening used may be the cross-section of the sample vessel that is open at the top or to be opened at the top. In further embodiments, the access opening may be formed by a separate channel ending in the cavity. The same applies to any outlet opening present.

In a further possible embodiment of the disclosure, only the sample space is partly or entirely covered by a lid, whereas at least a portion of the cross-section of the cavity (plan view) serving as an access opening remains free.

In order to create a region in the cavity for the delivery of the medium, for example by using a pipette tip guided into the cavity, a clearance space into which the medium can be delivered advantageously remains between the lateral wall of the sample space and a wall of the cavity. This clearance space advantageously serves as an access opening at least over sections of its extent. In the context of this description, delivery or withdrawal of the medium is usually performed by using pipette tips by way of example. Identical in meaning are corresponding conduits, tubing and/or channels.

The clearance space may run laterally around the sample space. In this case, the sample space is surrounded all around by the lateral walls and the clearance space. The sample space may be formed in the middle of the cavity, such that the clearance space between the sample space and the wall running around the cavity is approximately constant. Such an embodiment supports all-sided exchange or all-sided contacting of the media in the cavity and optionally uniform flow through the cavity and/or sample space.

In other embodiments of the sample vessel according to the disclosure, the clearance space may be present at least over a lateral angular range or sector of the sample space. The wall of the sample space is formed by the wall of the cavity over one section, whereas the lateral wall having the apertures delimits the remaining sector with respect to the cavity. This makes it possible to create a large clearance space.

In order to exchange a medium present in the cavity, an outlet opening through which the medium can be discharged out of the cavity may be present besides the access opening. The medium may be removed on the side open at the top, for example by contacting a (further) pipette tip there with the medium and removing a portion of the medium via an aspirator (pipette, pump) connected to the pipette tip.

In order to achieve uniform flow over and/or through the sample space, the outlet opening may be formed in or near the bottom of the cavity. When medium is delivered in an upper region of the cavity and the medium is discharged near the bottom, flow through the cavity and the sample space takes place, and remaining dead spaces in which little or no media exchange occurs are advantageously reduced.

The outlet opening may moreover have a closure to be operated in a controlled manner, in order to influence a rate of volume flow of the medium through the cavity in conjunction with the amount of medium delivered. The access opening may likewise be provided with a closure to be operated in a controlled manner.

The closure may be implemented, for example, in the form of a valve, a gate valve or a bladed shutter. When using pipette tips or the like, a controlled closure or a drive for operation of the closure is also considered to mean the corresponding technical elements of, for example, a pipette head, a Multipette or the like, viawhich the content of, for example, a pipette tip can be dispensed or drawn into the pipette tip.

The outlet opening may be especially formed at the bottom of the cavity together with an outlet nozzle. A conduit, for example, may be in connection with or may be connected to the latter.

Furthermore, it is possible that the outlet opening is closed by a porous matrix and that the flow resistance thereof prevents the medium from flowing out of the cavity. In order to discharge the medium, a negative pressure may be applied to the outlet opening, the effect of the negative pressure causing the medium to be drawn through the porous matrix. Such an embodiment allows the use of the sample vessel without having to establish an interlocking connection with a channel, tubing or the like. One of the ends of a channel could be pressed against the bottom of the sample vessel, with the channel end surrounding the outlet opening. Advantageously, present on an end face (channel end) of the channel that is pressed against the bottom is a seal to support the development of a negative pressure and to avoid any leakage of the medium beyond the channel. Instead of a porous matrix, in further embodiments of the sample vessel according to the disclosure, a closure may be made of a flexible material, for example a flap or a star-shaped closure made of a rubber compound.

In a simple embodiment of the sample vessel according to the disclosure, the at least one lateral wall stands on the bottom of the sample vessel. In one advantageous development, a sample support may for example be formed on the bottom of the sample space. Resting thereon may be the sample to be cultivated. The sample support may have a surface structure which, for example, serves for the adhesion of the sample or of constituents from which the sample will develop or further develop over a period of time. The surface structure may be a physical configuration of the surface, for example by having specific roughness values or having regular or irregular textures. The sample support may alternatively or additionally be provided with molecules (linkers) that allow specific binding of molecules of the samples or of molecules present in the medium to the sample support. In further embodiments of the disclosure, the sample space, in particular its bottom and/or at least one region of the lateral wall, may have such surface structures.

In addition to or as an alternative to the above possible embodiments, the shape of the sample support may cause effects on a sample. In one advantageous embodiment, the sample support is concavely curved in the direction of the sample space and thus forms a hollow. It has been found that such a shape supports the formation of spheroids, that is, aggregations of individual cells and/or cell clusters.

In order to allow better optical detection, visual monitoring and/or visual representation of the processes in the sample vessel, in particular in the sample space, the bottom of the sample vessel and/or the sample space may be formed by at least two planar or curved lateral walls that enclose an angle of less than 180°. Such an embodiment is advantageous for optical detection, visual monitoring and/or visual representation through the bottom of the sample vessel, that is, viaan inverted arrangement of illumination beam path and detection beam path. This advantageously reduces imaging errors that occur when illumination radiation and/or detection radiation pass(es) obliquely through the bottom of the sample vessel.

Advantageous for operation of a sample vessel of the disclosure according to one of the aforementioned embodiments is an apparatus for operating the sample vessel that includes a first pump for delivering the medium into the cavity or into the clearance space of the cavity. The first pump is connected to a controller in such a way as to allow exchange of data and transmission of control commands. The controller may be, for example, a computer, a microcontroller or an FPGA (field-programmable gate array).

Accordingly, in a further embodiment, a second pump serving for discharge of the medium through the outlet opening may be present. The second pump is likewise advantageously controlled by the controller.

The apparatus for operating the sample vessel may be part of a microscope. The latter includes a light source for providing illumination radiation. It is guided along an illumination beam path and may optionally be shaped by optical elements arranged therein, for example optical lenses. Furthermore, the microscope includes a detection objective for detecting detection radiation coming out of the sample space and includes a detector for converting detected detection radiation into electronic signals (image data).

The light source and the detection objective may be configured for transmitted light illumination. The sample space and the sample present therein are illuminated by the illumination radiation. The detection radiation used may be reflected and/or attenuated illumination radiation. The action of the illumination radiation may also trigger the emission of a detection radiation in the sample, for example through labeling of constituents of the sample with fluorophores (markers) that can be excited to emit fluorescent light by the illumination radiation.

Transmitted light illumination may be used especially for quantitative assessment of the processes in the sample space or of the current properties of the sample.

In a further embodiment of a microscope according to the disclosure, the light source and the detection objective are configured for inverted illumination and detection through a bottom of the sample vessel.

In further possible uses, the apparatus for operating the sample vessel may be part of an arrangement of an imaging method that does not involve direct illumination of the sample. For example, such an arrangement may be configured for optical coherence tomography. The sample vessel according to the disclosure can thus be used not only in light microscopy methods, but also in other imaging methods, for example scanning methods and/or under illumination of the sample with invisible light.

The abovementioned possible embodiments of the sample vessel may also be realized mutatis mutandis if more than one sample space is formed in a cavity. In such a case, multiple samples can, for example, interact via the medium surrounding the samples and occupying the cavity and, for example, exchange messengers and growth factors without the samples directly touching each other. Such a sample vessel may have, for example, a base and the dimensions of a standardized support, for example a standard plate in laboratory operation, for example an SBS plate.

Such an embodiment of the disclosure makes it possible, for example, to cultivate multiple samples, even different samples, such as organoids in a sample vessel and to allow chemical communication between the samples.

On the other hand, it is advantageously possible to arrange a plurality of sample vessels on a common support. Such a support may advantageously have the dimensions of standardized plates, for example SBS plates or the like, and have, for example, 6, 12, 24, 48, 96 or 384 sample vessels. This allows the disclosure to be used with already existing laboratory equipment and, if necessary, to be automated with ease.

If there are multiple sample vessels per support, they may be placed on a common base plate which forms the respective bottoms of the sample vessels.

The sample supports according to the disclosure may of course be advantageously provided in sterilized form.

### BRIEF DESCRIPTION OF DRAWINGS

The invention will now be described with reference to the drawings wherein: FIG. 1 shows a schematic illustration of a first embodiment of a sample vessel according to the disclosure in a perspective view in the form of a wire model; FIG. 2 shows a schematic illustration of a second embodiment of a sample vessel according to the disclosure in a perspective view in the form of a wire model;

FIG. 3 shows a schematic illustration of a third embodiment of a sample vessel according to the disclosure in a perspective view in the form of a wire model;

FIG. 4 shows a schematic illustration of a first embodiment of an apparatus according to the disclosure and a fourth embodiment of a sample vessel according to the disclosure in a lateral sectional view;

FIG. 5 shows a schematic illustration of the first embodiment of an apparatus according to the disclosure and a fifth embodiment of a sample vessel according to the disclosure in a lateral sectional view;

FIG. 6 shows a schematic illustration of a second embodiment of an apparatus according to the disclosure and a sixth embodiment of a sample vessel according to the disclosure in a lateral sectional view;

FIG. 7 shows a schematic illustration of a third embodiment of an apparatus according to the disclosure in a lateral sectional view;

FIG. 8 shows a schematic illustration of a first embodiment of a microscope according to the disclosure as a transmitted light microscope in a lateral sectional view;

FIG. 9 shows a schematic illustration of the first embodiment of a microscope according to the disclosure as a transmitted light microscope and a seventh embodiment of a sample vessel according to the disclosure having a concave sample support in a lateral sectional view;

FIG. 10 shows a schematic illustration of a second embodiment of a microscope according to the disclosure as an inverted microscope in a lateral sectional view;

FIG. 11 shows a schematic illustration of the second embodiment of a microscope according to the disclosure as an inverted microscope and an eighth embodiment of a sample vessel according to the disclosure in a lateral sectional view;

FIG. 12 shows a schematic illustration of the second embodiment of a microscope according to the disclosure as an inverted microscope and a ninth embodiment of a sample vessel according to the disclosure in a lateral sectional view;

FIG. 13 shows a schematic illustration of a support in the form of a plate having a plurality of sample vessels according to the disclosure in a perspective view; and,

FIG. 14 shows a schematic illustration of a sample vessel according to the disclosure in the form of a support having a plurality of sample spaces in fluidic communication with each other via a medium.

### DETAILED DESCRIPTION

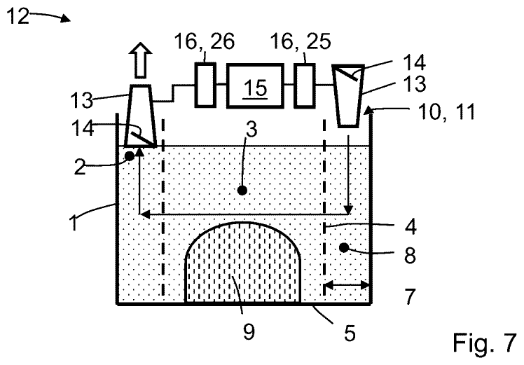

The drawings of the embodiments are schematic and not true to scale. For better visualization, FIGS. 1 to 3 are shown as so-called wire models, which show only the outer contours of the respective structures and dispense with showing closed surfaces, for example the wall of the sample vessel 1. In FIGS. 7 to 12, technical elements used for delivering and removing a medium 8 are omitted for the sake of clarity.

In the embodiment shown, a sample vessel 1 according to the disclosure is in the form of a hollow cylinder that is open at the top and has a bottom 5 (FIG. 1). The inner volume of the sample vessel 1, which is referred to as cavity 2, accommodates a sample space 3, at least one lateral wall 4 of which has a plurality of apertures 6 and stands on the bottom 5. In all embodiments, the bottom 5 is transparent at least over the extent of the sample space 3 and at least for specific wavelength ranges, in particular of visible and/or infrared light.

The sample space 3 has a diameter smaller than the diameter of the cavity 2, such that a clearance space 7 remains between the lateral wall 4 and the wall of the sample vessel 1. In the first embodiment, the sample space 3 is disposed centrally in the cavity 2, such that the clearance space 7 runs around the sample space 3 at a constant extent. The clearance space 7 serves as an access opening 10 and/or as an outlet opening 11.

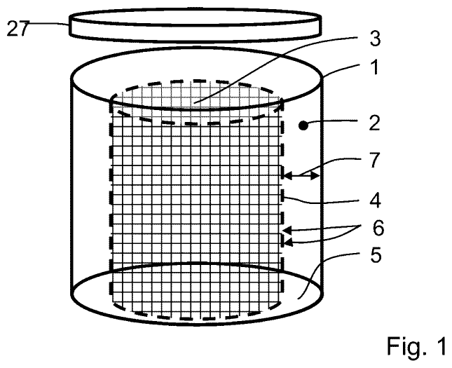

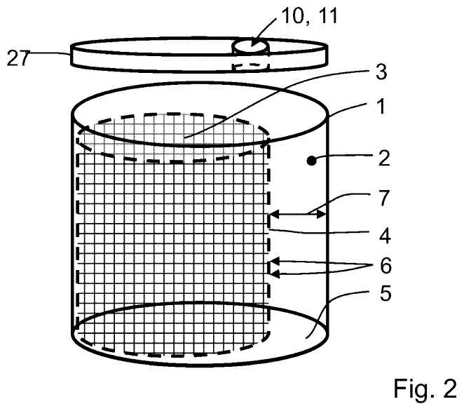

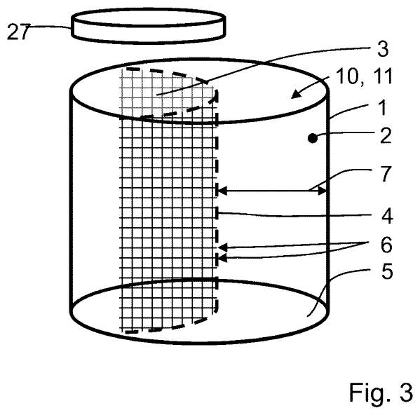

Optionally present is a lid 27 viawhich the sample vessel 1 can be closed, but at least covered if necessary. In further embodiments, the lid 27 may also cover only a portion of the cavity 2 (see FIG. 3). Furthermore, the lid 27 may have openings for delivering and/or removing a medium 8 present therein (see also FIG. 2). The openings may be actively or passively openable or reclosable.

In a second embodiment, the sample space 3 is disposed off-center in the cavity 2, thus creating, in one direction, a larger clearance space 7 compared to the first embodiment despite the sample space 3 being of identical size (FIG. 2). In the variant embodiment shown, the optionally present lid 27 has an access opening 10 through which medium 8 can be introduced into the clearance space 7. The opening 10 may of course, as desired, also serve as an outlet opening 11 through which the entire medium 8 or a portion thereof can be withdrawn.

In a third embodiment, the lateral wall 4 may extend from one especially vertically extending contact line on the wall of the sample vessel 1 to another vertical contact line on the wall, and may be joined to the wall at both contact lines (FIG. 3). Here, the lid 27 to be optionally used closes, for example, only the top of the sample space 3, whereas the clearance space 7 remains substantially freely accessible from above and serves as an access opening 10 and/or outlet opening 11.

Taking into consideration the technical requirements, the embodiments of the lid 27 may be freely combined with the embodiments of the sample vessel 3 described above or below.

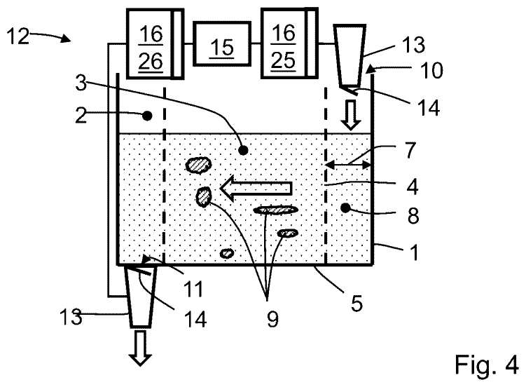

The sample vessel 1 may be used in an apparatus 12 for operating the sample vessel 1. FIG. 4 shows, by way of example, a vertical section of a sample vessel 1 partly filled with a medium 8. Floating in the medium 8 within the sample space 3 are individual cells, cell clusters and/or aggregations that serve as sample 9. The dimensions of the apertures 6 (see above) in the lateral wall 4 are such that the sample 9 itself is not flushed out of the sample space 3 when there is flow in the medium 8.

Present for delivery of the medium 8 to the cavity 2 is a conduit 13 in the form of, for example, a pipette tip directed into the clearance space 7 at the open side of the sample vessel 1 acting as an access opening 10. Starting from the conduit 13, the medium 8 can flow along the clearance space 7 and through the apertures 6 into and out of the sample space 3. The generation of a directed flow (indicated by arrows) is made possible by an outlet opening 11 being present in the region of the clearance space 7 and by an optional closure 14 used for closing the outlet opening 11 as desired being open. The outflowing medium 8 exits the sample vessel 1 again through a conduit 13 in the form of, for example, a connection nozzle for connecting tubing or for connecting a tube. The medium 8 is delivered at the access opening 10 viaa first pump 25 of the conduit 13 and pumped out via the outlet opening 11 viaa second pump 26.

Present for operation of the apparatus 12, in particular for control of the closures 14 and the pumps 25, 26, is a controller 15 that is connected to a drive 16 in each case. As already discussed above, a technical unit for dispensing a quantity of the medium 8 is also understood to be covered by the term “controllable closure”. The relevant drive 16 receives control commands from the controller 15 and, on executing the control commands, adjusts the degree of opening of the respective closure 14. A delivery rate of the medium 8 can be adjusted and controlled by control of the pumps 25, 26.

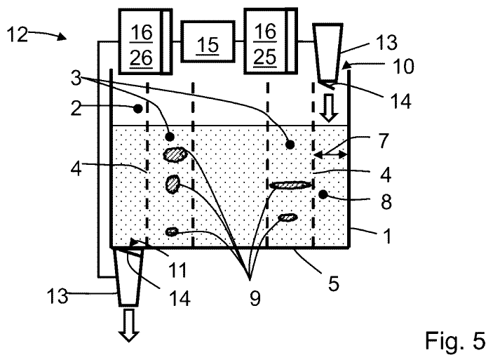

Shown on the basis of the first embodiment of the apparatus 12 according to the disclosure is a fifth embodiment of the sample vessel 1 according to the disclosure, in which a plurality of sample spaces 3, in this case two, are present in the cavity 2 (FIG. 5). They are in fluidic communication with each other. Such an embodiment of the sample vessel 1 may be used, for example, when reactions of the sample 9 in a fluidically downstream sample vessel 1 is to be tested for, for example, physiological processes in a fluidically upstream sample vessel 1. For example, hormones, growth factors and/or metabolites may be transported with the flowing medium 8 from the upstream sample space 3 to the downstream sample space 3, where they may possibly trigger a reaction.

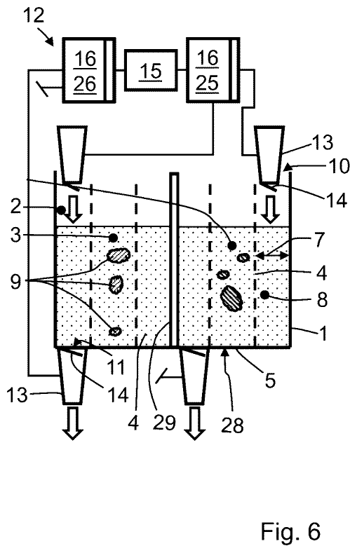

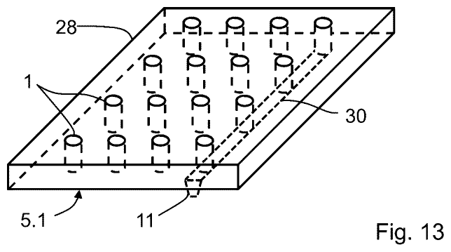

The apparatus 12 according to the disclosure may be used with a further embodiment of the sample vessel 1 according to the disclosure (FIG. 6). Formed on a support 28 are multiple sample spaces 3. Each of the sample spaces 3 is laterally delimited with respect to its neighboring sample spaces 3 by a partition wall 29, the partition wall 29 preventing exchange of liquid or gaseous media. Each of the sample spaces 3 may undergo individually controlled filling via a conduit 13 and emptying via an outlet opening 11. In further embodiments, a common conduit 13 may be used. To this end, the support 28 and the conduit 13 undergo controlled movement relative to each other, for example through the presence and use of a pipetting head and/or a movable sample table. The relative movement may likewise be controlled and closed loop-controlled or open loop-controlled via the controller 15. Further alternatives have a common outlet channel 30 (see FIG. 13) via which medium can be withdrawn from some or all of the sample vessels 1.

In a second embodiment of an apparatus 12 according to the disclosure (FIG. 7), the side of the sample vessel 1 open at the top is used both as an access opening 10 for a conduit 13 and as an outlet opening 11. The sample 9 is, by way of example, an organoid or spheroid having a three-dimensional extent.

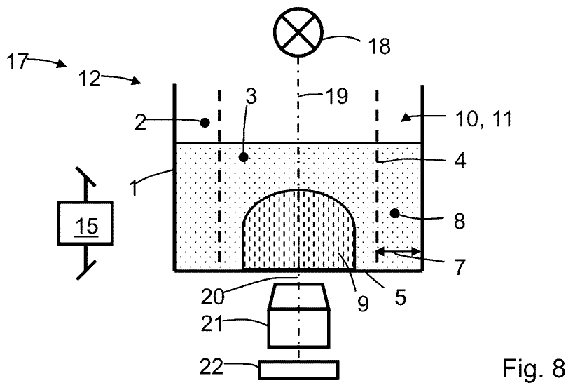

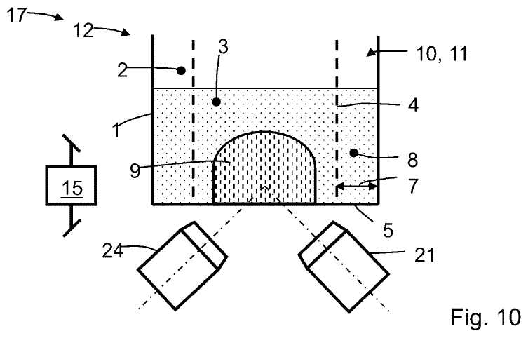

The apparatus 12 may be part of a microscope 17 configured for transmitted light illumination (FIG. 8). The microscope 17 includes a light source 18 which provides illumination radiation which is directed along an illumination beam path 19 into the sample space 3. Present in the illumination beam path 19 for the purpose of conducting and/or shaping the illumination radiation may be optical elements, for example an illumination objective 24 (see for example FIG. 10), which are not shown here for the sake of clarity. The components of the illumination radiation passing through the sample 9 and/or fluorescence radiation emitted as a result of the action of the illumination radiation are guided along a detection beam path 20, collected viaa detection objective 21, steered to a detector 22 and detected thereby as measured values (image signals, image data).

In the embodiment, the sample space 3 and a sample 9 present therein are illuminated from above the sample vessel 1. In further embodiments not shown, transmitted light illumination may be achieved by arrangement of the light source 18 below the sample vessel 1 and illumination through the transparent bottom 5. In such an embodiment, the detection objective 21 and the detector 22 may be above the sample space 3. Besides the functions of the controller 15 that have already been described above, it may also control the light source 18 and/or the detector 22.

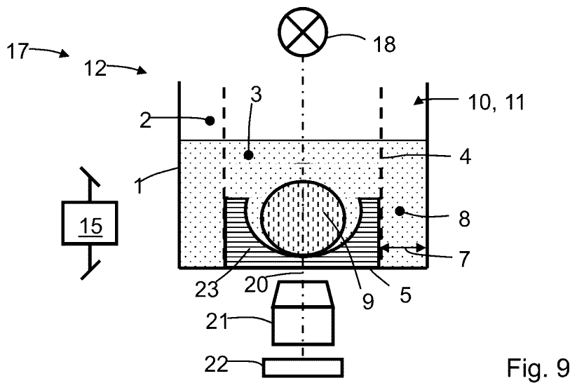

In a fifth embodiment of a sample vessel 1 according to the disclosure, it has a concave sample support 23 on its bottom 5 (FIG. 9). The shape and dimensioning of the sample support 23 advantageously supports aggregation of, for example, cells and relatively small cell clusters to give a spheroid forming the sample 9. The sample support 23 is transparent for detection radiation to be detected and/or illumination radiation. Any light-scattering effect of the sample support 23 is harmless if, for example, only the presence of, for example, fluorescence radiation of a specific wavelength is to be detected as detection radiation, without aiming at imaging the sample 9 and/or locating the site of origin of the detection radiation. The material of the bottom 5 and of the sample support 23 has advantageously a high degree of transparency (transmissivity) for the detection radiation wavelengths to be used. A working distance between the detection objective 21 and a sample 9 to be detected is advantageously kept as short as possible.

A microscope 17 having an inverted illumination and detection arrangement is shown in FIG. 10. Illumination is achieved viaan illumination objective 24 directed to the bottom 5 from outside the sample vessel 1 at an angle not equal to 90°, advantageously at an angle in a range from 30° to 60°. The detection radiation is detected via the detection objective 21 that is likewise directed to the bottom 5 from outside the sample vessel 1 at an angle in a range from 30° to 60°, the optical axes of the illumination objective 24 and the detection objective 21 advantageously mutually enclosing an angle of 90°.

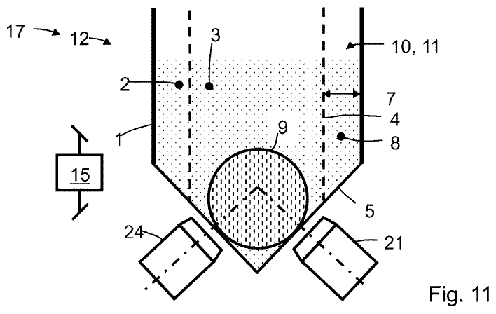

In a further embodiment of the sample vessel 1 according to the disclosure in combination with the inverted microscope 17, in order to reduce the aberrations that occur when the illumination and detection radiation pass obliquely through the bottom 5, the bottom 5 may advantageously be formed by two or more walls mutually enclosing an angle not equal to 180° (FIG. 11). The illumination objective 24 and the detection objective 21 are arranged in relation to the bottom 5 such that their respective optical axes are directed perpendicularly to the relevant wall of the bottom 5. This can avoid a considerable proportion of the aberrations that occur at the interface of the bottom 5. At the same time, the shape of the walls of the bottom 5 can advantageously support the formation of spheroids.

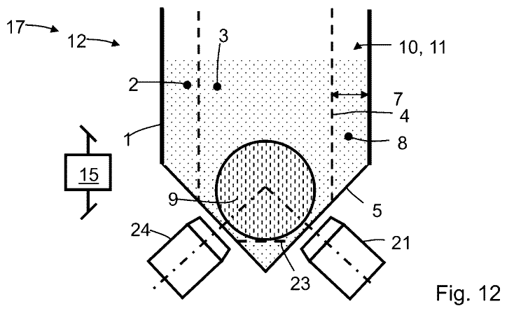

A modification of the sample vessel 1 shown in relation to FIG. 11 has a sample support 23 above the lowest point of the sample space 3 (FIG. 12). It is used to position the sample 9 relative to the beam paths of the objectives 21, 24. Just like that of the example described above, the sample support 23 may be provided with a surface structure beneficial for adhesion and/or aggregation. The inverted embodiments of the microscope 17 may of course also be used for examining free-floating samples 9.

FIG. 13 provides a simplified illustration of a support 28 in the form of a plate containing a plurality of sample vessels 1 (shown in simplified form) arranged in rows and columns. The sample vessels 1 are open on the face of the support 28 that is facing upward. A common base plate 5.1 of the support 28 closes the lower side of the sample vessels 1 and forms the bottom 5 (see above) of the respective sample vessel 1. The base plate 5.1 consists of a material transparent for detection radiation and/or illumination radiation. The support 28 may consist of a single material. In further embodiments, the base plate 5.1 is made of a material different from the rest of the support 28.

The sample vessels 1 may be optionally and individually provided with a lid 27. In a further possible embodiment, a common lid 27 (not shown) with or without access opening 10 and covering, if necessary, some or all of the sample vessels 1 present may be present.

The individual sample vessels 1 each have an outlet opening 11 in the base plate 5.1 or in the face of the support 28 shown facing upward. It is possible for the outlet openings 11 of all the sample vessels 1, or of a number thereof, to be connected to an outlet channel 30 via which the medium 8 can be withdrawn from the relevant sample vessels 1 (shown as an option by a broken solid line). A closure 14 of the sample vessels 1 may be optionally present. Accordingly, a common closure 14 (not shown) of the outlet channel 30 may be present.

An outlet channel 30 may, for example, connect the sample vessels 1 arranged on the support 28 in a row and/or in a column. In further embodiments, selected sample vessels 1 may be connected to each other in a different arrangement.

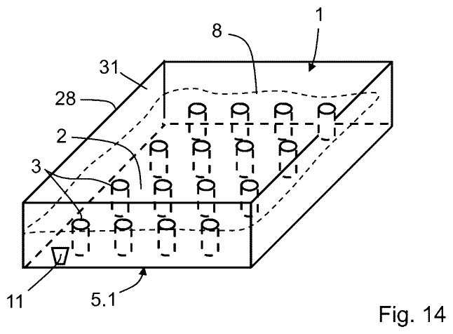

In a further embodiment of a support 28, it is in the form of a sample vessel 1, by having a base plate 5.1 with a peripheral wall 31 standing on the base plate 5.1 (FIG. 14). Disposed within a cavity 2 surrounded by the wall 31 are a plurality of sample spaces 3 standing on the base plate 5.1. When the cavity 2 has been filled with a medium 8, the sample spaces 3 are fluidically connected to each other thereby. Via an outlet opening 11, the medium 8 may be introduced into the cavity 2 and withdrawn therefrom. Here too, a lid 27 may be optionally present, optionally having moreover an access opening 10. This embodiment is advantageous in that different cell types or organoid types can be cultivated in the different sample spaces 3 and they can exchange substances via the common medium 8 without themselves coming into direct contact.

It is understood that the foregoing description is that of the preferred embodiments of the invention and that various changes and modifications may be made thereto without departing from the spirit and scope of the invention as defined in the appended claims.

### REFERENCE SIGNS

1 Sample vessel
2 Cavity
3 Sample space
4 Lateral wall
5 Bottom
5.1 Base plate
6 Aperture
7 Clearance space
8 Medium
9 Sample
10 Access opening
11 Outlet opening
12 Apparatus
13 Pipette tip, conduit, tubing connector
14 Closure
15 Controller
16 Drive
17 Microscope
18 Light source
19 Illumination beam path
20 Detection beam path
21 Detection objective
22 Detector
23 Sample support
24 Illumination objective
25 First pump unit
26 Second pump unit
27 Lid
28 Support
29 Partition wall
30 Outlet channel
31 Wall

1. A sample vessel for cultivating biological samples, the sample vessel comprising:
a cavity for accommodating a medium;
the sample vessel defining at least one access opening for delivering the medium into said cavity;
at least one sample space, disposed within said cavity, for accommodating a sample, said at least one sample space being separated from a remaining space of said cavity by at least one lateral wall and the at least one lateral wall defining apertures via which the medium present in said cavity can communicate with said sample space;
wherein said at least one lateral wall stands on a bottom of said sample space; and,
said bottom is transmissive for wavelengths of at least one wavelength range of visible light such that at least one of illumination of said sample space and detection of detection radiation coming out of said sample space through said bottom is enabled.

2. The sample vessel of claim 1, wherein said apertures have a maximum internal width of at most 1000 μm.

3. The sample vessel of claim 1, wherein a clearance space into which the medium is deliverable remains between said lateral wall of said sample space and a wall of said cavity.

4. The sample vessel of claim 3, wherein said clearance space runs around said sample space.

5. The sample vessel of claim 3, wherein said clearance space is present at least over a lateral angular range of said sample space.

6. The sample vessel of claim 1, wherein said cavity defines an outlet opening through which the medium is dischargeable out of said cavity.

7. The sample vessel of claim 6, wherein said outlet opening has a closure to be operated in a controlled manner.

8. The sample vessel of claim 6, wherein said outlet opening is provided with a porous matrix through which the medium can be drawn out of said cavity via a generated negative pressure on the side facing away from said sample space.

9. The sample vessel of claim 1 further comprising a sample support disposed at said bottom of said sample space and being concavely curved in a direction of said sample space.

10. The sample vessel of claim 1, wherein at least one of said bottom of said sample space and said sample space is formed by at least two planar walls that enclose an angle of less than 180°.

11. The sample vessel of claim 1, wherein said apertures have a maximum external width of at most of at most 500 μm.

12. The sample vessel of claim 1, wherein said apertures have a maximum external width of at most of at most 200 μm.

13. An apparatus for operating a sample vessel including a cavity for accommodating a medium, the sample vessel defining at least one access opening for delivering the medium into the cavity, the sample vessel further including at least one sample space, disposed within the cavity, for accommodating a sample, the at least one sample space being separated from a remaining space of the cavity by at least one lateral wall and the at least one lateral wall defining apertures via which the medium present in the cavity can communicate with the sample space, wherein the at least one lateral wall stands on a bottom of the sample space, and, the bottom is transmissive for wavelengths of at least one wavelength range of visible light such that at least one of illumination of the sample space and detection of detection radiation coming out of the sample space through the bottom is enabled, the apparatus comprising:
a first pump unit for delivering the medium into said cavity; and
a controller for controlling said first pump unit.

14. The apparatus of claim 11 further comprising:
a second pump unit;
wherein the cavity defines an outlet opening; and,
said second pump unit being configured to discharge the medium through the outlet opening, said second pump unit being controlled via said controller.

15. A microscope comprising:
a light source for providing illumination radiation along an illumination beam path;
an apparatus for operating a sample vessel including a cavity for accommodating a medium, the sample vessel defining at least one access opening for delivering the medium into the cavity, the sample vessel further including at least one sample space, disposed within the cavity, for accommodating a sample, the at least one sample space being separated from a remaining space of the cavity by at least one lateral wall and the at least one lateral wall defining apertures via which the medium present in the cavity can communicate with the sample space, wherein the at least one lateral wall stands on a bottom of the sample space, and, the bottom is transmissive for wavelengths of at least one wavelength range of visible light such that at least one of illumination of the sample space and detection of detection radiation coming out of the sample space through the bottom is enabled;
said apparatus including a first pump unit for delivering the medium into said cavity and a controller for controlling said first pump unit;
a detection objective for detecting detection radiation coming out of the sample space; and,
a detector for converting detected detection radiation into electronic signals.

16. The microscope of claim 15, wherein said light source and said detection objective are configured for transmitted light illumination.

17. The microscope of claim 15, wherein said light source and said detection objective are configured for inverted illumination and detection through the bottom of the sample space.
